(19) United States
(12) Patent Application Publication
(10) Pub. No.: US 2004/0242891 A1
(43) Pub. Date: Dec. 2, 2004

(54) Production of keto acids

(76) Inventors: Jonathan Campbell, Oldham (GB); John Barry Henshall, Manchester (GB); James Philip Taylor, Oldham (GB); John Whitworth, Manchester (GB)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/492,467

(22) PCT Filed: Oct. 17, 2002
(86) PCT No.: PCT/EP02/11647

(30) Foreign Application Priority Data
Oct. 26, 2001 (EP) 01811051.0

(51) Int. Cl. C07D 211/34; C07D 207/04; C07C 229/52

(52) U.S. Cl. 546/238; 548/577; 562/452

(57) ABSTRACT

An improved method of producing keto acids having the formula (I), wherein R1 and R2 independently represent (a) hydrogen,

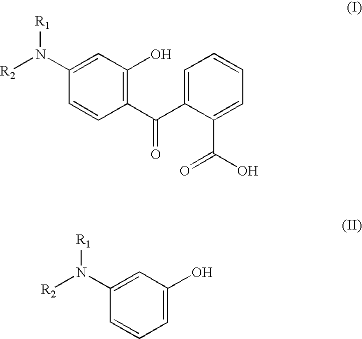

wherein at least one of R1 and R2 do not stand for hydrogen, (b) branched or unbranched alkyl of 1-18 carbon atoms, which may be substituted by C1-C4alkoxy or 2- or 3-tetrahydrofuryl, (c) a cycloalkyl of 4-8 carbon atoms, (d) C4-C8cycloalkyl-C1-C4alkyl, or phenyl, wherein both, cycloalkyl and phenyl, may be substituted by at least one member selected from the group consisting of halogen atoms and alkyls having 1-4 carbon atoms, (e) an aralkyl of 7-10 carbon atoms, or (f) R1 and R2 together with the adjacent nitrogen atom may form a heterocyclic ring, by reacting a m-amino phenol having the formula (II) with phthalic anhydride at an elevated temperature in the absence of an organic solvent, which comprises: (I) mixing m-amino phenol II and phthalic anhydride in a molar ratio of from 0.5 to 10:1, (II) melting the mixture of step I at an elevated temperature, (III) choosing a reaction time in the range of from 5 minutes to 40 hours, (IV) then separating the liquid phase from the solid phase as well as a method in which a solvent is added after the reaction.

The invention relates to an improved method of producing keto acids having the formula I

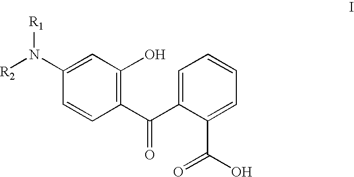

wherein R₁ and R₂ independently represent

(a) hydrogen, wherein at least one of R₁ and R₂ do not stand for hydrogen,

(b) branched or unbranched alkyl of 1-18 carbon atoms, which may be substituted by C₁-C₄alkoxy or 2- or 3-tetrahydrofuryl,

(c) a cycloalkyl of 4-8 carbon atoms,

(d) C₄-C₈cycloalkyl-C₁-C₄alkyl, or phenyl, wherein both, cycloalkyl and phenyl, may be substituted by at least one member selected from the group consisting of halogen atoms and alkyls having 1-4 carbon atoms,

(e) an aralkyl of 7-10 carbon atoms, or

(f) R₁ and R₂ together with the adjacent nitrogen atom may form a heterocyclic ring,

by reacting a m-amino phenol having the formula II

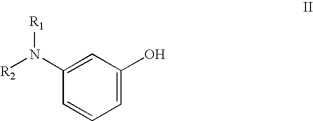

with phthalic anhydride at an elevated temperature in the absence of an organic solvent, which comprises:

(I) mixing m-amino phenol II and phthalic anhydride in a molar ratio of from 0.5 to 10:1, preferably from 1:1 to 3:1,

(II) melting the mixture of step I at an elevated temperature,

(III) choosing a reaction time in the range of from 5 minutes to 40 hours,

(IV) then separating the liquid phase from the solid phase.

Such keto acids are useful intermediates for the production of fluoran compounds used in pressure- or heat-sensitive recording materials.

German patent no. 87068 dated Mar. 03, 1895 describes a process in which an m-amino phenol and phthalic anhydride are reacted in a melt at 100° C. for several hours without any solvent. After the reaction the obtained solid is dissolved in ethanol. After filtration, water is added to the hot solution thus initiating the precipitation of the desired keto acid. This process has the disadvantage that the obtained solid has to be pulverized before it can be further worked-up, which is highly unfavorable in nowadays-industrial processes.

EP-A 511,019 describes a method of producing a keto acid which comprises reacting a m-amino phenol with phthalic anhydride in the presence of an organic solvent, the organic solvent being present in an amount of 0.5 to 3 parts by weight per one part by weight of the m-amino phenol with the effect that the resultant keto acid is deposited in the solvent so that the reaction is effected in a slurry.

The amount of organic solvent used can cause loss of yield due to the solubility of the product keto acid in the organic solvent. The disposal of large amounts of organic solvent poses significant economic and ecological problems. In addition, extended reaction times are often required for processes that are affected in the presence of an organic solvent.

Therefore, an object of this invention was to provide an improved method of producing keto acids in the absence of an organic solvent, which avoids the abovementioned disadvantages. In particular, a process should be provided in which the rhodamine amount can be decreased or even eliminated, and/or in which the yield could be increased.

Accordingly, the above-described method was found.

The m-amino phenols used in the invention are known or can be prepared according to known methods.

Alkyl (which may or may not be branched) of 1-18 carbon atoms stands for methyl, ethyl, n-, i-propyl, n-, i-, sec.-, tert.-butyl, n-pentyl, isoamyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosyl, preferably C₁-C₈alkyl such as methyl, ethyl, n-, i-propyl, n-, i-, sec.-, tert.-butyl, n-pentyl, n-hexyl, n-heptyl, n-octyl, more preferably for C₁-C₄alkyl such as methyl, ethyl, n-, i-propyl, n-, i-, sec.-, tert.-butyl;

alkyls having 1-4 carbon atoms stands for methyl, ethyl, n-, i-propyl, n-, i-, sec.-, tert.-butyl; cycloalkyl of 4-8 carbon atoms stands for cyclobutyl, cyclopentyl, cyclohexyl, cycloheptyl, cyclooctyl;

halogen stands for fluorine, chlorine, bromine, iodine,

aralkyl of 7-10 carbon atoms stands for benzyl, 2-phenylethyl, 3-phenylpropyl, 4-phenylbutyl,

C₄-C₈cycloalkyl-C₁-C₄alkyl such as cyclobutylmethyl, cyclopentylmethyl, cyclohexylmethyl cyclopentylethyl, cyclohexylethyl cyclopentylpropyl, cyclohexylpropyl, cyclopentylbutyl, cyclohexylbutyl, cycloheptylmethyl, cyclooctylmethyl;

if R₁ and R₂ together with the adjacent nitrogen atom form a heterocyclic ring, then such a heterocyclic ring may be 2-, 3-, or 4-pyridyl, pyrazinyl, 3-isooxazolyl, 1-pyrazolyl, 3-pyrrolyl, 2H-pyrrol-3-yl, 3-pyrazolin-2-yl, 2-piperidyl, 2-piperazinyl, 1-indolinyl, 3-morpholinyl, 2- or 3-pyrrolidinyl.

Preferred m-amino phenols are N,N-di-methyl aminophenol, N,N-di-ethyl aminophenol, N-methyl-N-ethyl aminophenol, N,N-di-n-propyl aminophenol, N,N-di-n-butyl aminophenol, N,N-di-n-pentyl aminophenol, N,N-di-n-hexyl aminophenol, N,N-diisopropyl aminophenol, N,N-diisobutyl aminophenol, N,N-disecbutyl aminophenol, N,N-diisoamyl aminophenol, N-ethyl-N-cyclohexyl aminophenol, N-ethyl-N-isoamyl phenol, N-ethyl-N-cyclohexylmethyl aminophenol, N-phenyl-N-ethyl aminophenol, 3-N-pyrrolidinyl phenol, N-methyl-N-cyclohexyl aminophenol, N-methyl-N-phenyl aminophenol, N-methyl-N-(2-methylphenyl)aminophenol, N-methyl-N-(3-methylphenyl)aminophenol, N-methyl-N-(4-methylphenyl)aminophenol, N-methyl-N-propyl aminophenol, N-methyl-N-isopropyl aminophenol, N-methyl-N-butyl aminophenol, N-methyl-N-isobutyl aminophenol, N-methyl-N-sec.butyl aminophenol, N-methyl-N-pentyl aminophenol, N-methyl-N-1-methylbutyl aminophenol, N-methyl-N-isoamyl phenol, N-methyl-N-1-methylpentyl aminophenol, N-methyl-N-hexyl aminophenol, N-methyl-N-tetrahydrofurylmethyl aminophenol, N-methyl-N-ethoxypropyl aminophenol, N-methyl-N-cyclohexylmethyl aminophenol, N-methyl-N-phenethyl aminophenol, N-ethyl-N-phenyl aminophenol, N-ethyl-N-(2-methylphenyl)aminophenol, N-ethyl-N-(3-methylphenyl)aminophenol, N-ethyl-N-(4-methylphenyl)aminophenol, N-ethyl-N-propyl aminophenol, N-ethyl-N-isopropyl aminophenol, N-ethyl-N-butyl aminophenol, N-ethyl-N-isobutyl aminophenol, N-ethyl-N-secbutyl aminophenol, N-ethyl-N-pentyl aminophenol, N-ethyl-N-1-methylbutyl aminophenol, N-ethyl-N-isoamyl phenol, N-ethyl-N-1-methylpentyl aminophenol, N-ethyl-N-hexyl aminophenol, N-ethyl-N-tetrahydrofurylmethyl aminophenol, N-ethyl-N-ethoxypropyl aminophenol, N-ethyl-N-cyclohexylmethyl aminophenol, N-ethyl-N-phenethyl aminophenol, N-propyl-N-cyclohexyl aminophenol, N-propyl-N-phenyl aminophenol, N-propyl-N-(2-methylphenyl)aminophenol, N-propyl-N-(3-methylphenyl)aminophenol, N-propyl-N-(4-methylphenyl)aminophenol, N-propyl-N-isopropyl aminophenol, N-propyl-N-butyl aminophenol, N-propyl-N-isobutyl aminophenol, N-propyl-N-sec.butyl aminophenol, N-propyl-N-pentyl aminophenol, N-propyl-N-1-methylbutyl aminophenol, N-propyl-N-isoamyl aminophenol, N-propyl-N-1-methylpentyl aminophenol, N-propyl-N-hexyl aminophenol, N-propyl-N-tetrahydrofurylmethyl aminophenol, N-propyl-N-ethoxypropyl aminophenol, N-propyl-N-cyclohexylmethyl aminophenol, N-propyl-N-phenethyl aminophenol, N-butyl-N-cyclohexyl aminophenol, N-butyl-N-phenyl aminophenol, N-butyl-N-(2-methylphenyl)aminophenol, N-butyl-N-(3-methylphenyl)aminophenol, N-butyl-N-(4-methylphenyl)aminophenol, N-butyl-N-propyl aminophenol, N-butyl-N-isopropyl aminophenol, N-butyl-N-isobutyl aminophenol, N-butyl-N-secbutyl aminophenol, N-butyl-N-pentyl aminophenol, N-butyl-N-1-methylbutyl aminophenol, N-butyl-N-isoamyl phenol, N-butyl-N-1-methylpentyl aminophenol, N-butyl-N-hexyl aminophenol, N-butyl-N-tetrahydrofurylmethyl aminophenol, N-butyl-N-ethoxypropyl aminophenol, N-butyl-N-cyclohexylmethyl aminophenol, N-butyl-N-phenethyl aminophenol, N-phenyl aminophenol, N-2-methylphenyl aminophenol, N-3-methylphenyl aminophenol, N-4-methylphenyl aminophenol, N-cyclohexyl aminophenol, 3-N-pyrrolidinyl phenol, 3-N-(2-methylpyrrolidinyl)phenol, 3-N-(3-methylpyrrolidinyl)phenol, 3-N-morpholinyl phenol, 3-N-piperidinyl phenol, 3-N-(2-methylpiperidinyl)phenol, 3-N-(3-methylpiperidinyl)phenol, 3-N-(4-methylpiperidinyl)phenol.

Particularly preferred keto acids I are N,N-dibutylamino-2-hydroxy-2′-carboxybenzophenone, N,N-diethylamino-2-hydroxy-2′-carboxybenzophenone, N,N-dimethylamino-2-hydroxy-2′-carboxybenzophenone, N-isoamyl-N-ethyl amino-2-hydroxy-2′-carboxybenzophenone as well as N-propyl-N-methyl amino-2-hydroxy-2′-carboxybenzophenone, N-cyclohexyl-N-methyl amino-2-hydroxy-2′-carboxybenzophenone, N-4-methylphenyl-N-ethyl amino-2-hydroxy-2′-carboxybenzophenone, and N-isobutyl-N-ethyl amino-2-hydroxy-2′-carboxybenzophenone.

For the reaction of the m-aminophenol II with phthalic anhydride, the former is usually used in a molar ratio of 0.5:1 to 10:1, preferably from 1:1 to 3:1. Preferably, the amount of m-aminophenol II is chosen in such a way to ensure that the reaction product does not become solid at the reaction temperature. The amount usually depends on the m-aminophenolo II chosen. E.g. in case of dibutyl-m-aminophenol the ratio is in particular chosen in the range of from 1.3 to 1.5, particularly preferred 1.4. Other ratios are given in the examples.

In general, the reaction is effected at an elevated temperature, preferably in the range of from 60 to 170° C., more preferably from 80 to 110° C.

The reaction time usually is chosen in the range of from 5 minutes to 40 hours, preferably from 5 minutes to 12 hours, more preferably from 3 to 5 hours.

As a rule, the reaction time and temperature are chosen so as to achieve a suitable balance between length of reaction and the amount of rhodamine type side products that are produced.

In a preferred embodiment a reaction temperature in the range of from 80 to 110° C. and a reaction time in the range of from 3 to 5 hours are chosen.

After the reaction, the solid phase usually is removed from the reaction mixture, preferably by filtration. It may be preferable to reduce the reaction temperature to a range in between from 0 to 80, more preferably from 20 to 70° C. prior to this separation step (i.e. preferably filtration). In some cases filtration may be improved by addition of a diluent before or after this cooling step.

A preferred embodiment of this invention relates to a method of producing keto acids having the formula I

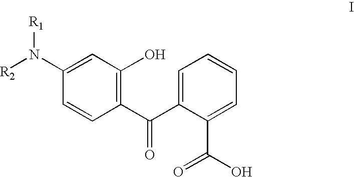

wherein R₁ and R₂ independently represent

(a) hydrogen, wherein at least one of R₁ and R₂ do not stand for hydrogen,

(b) branched or unbranched alkyl of 1-18 carbon atoms, which may be substituted by C₁-C₄alkoxy or 2- or 3-tetrahydrofuryl,

(c) a cycloalkyl of 4-8 carbon atoms,

(d) C₄-C₈cycloalkyl-C₁-C₄alkyl, or phenyl, wherein both, cycloalkyl and phenyl, may be substituted by at least one member selected from the group consisting of halogen atoms and alkyls having 1-4 carbon atoms,

(e) an aralkyl of 7-10 carbon atoms, or

(f) R₁ and R₂ together with the adjacent nitrogen atom may form a heterocyclic ring,

by reacting a m-amino phenol having the formula II

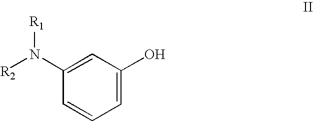

with phthalic anhydride at an elevated temperature in the absence of an organic solvent, which comprises the following reaction cycle:

(A) mixing m-amino phenol II and phthalic anhydride in a molar ratio of from 0.5 to 10:1, preferably from 1:1 to 3:1,

(B) melting the mixture of step (A) to an elevated temperature,

(C) choosing a reaction time in the range of from 5 minutes to 40 hours,

(D) adjusting the temperature of the reaction mixture to one suitable for effective separation,

(E) then separating the liquid phase from the solid phase, optionally washing the solid phase comprising keto acid I with an organic solvent and then drying it,

(F) adding phthalic anhydride and/or m-amino phenol II to the separated liquid phase of step (E), wherein the molar ratio of from 0.5 to 10:1, preferably from 1:1 to 3:1,

(G) using the obtained mixture of step (F) as starting material or as part of starting material of step (B) after removing the diluent,

wherein either after step C, but before step D or after step D, but before step E a diluent is added to the reaction mixture.

For the reaction of the m-aminophenol II with phthalic anhydride, the former is usually used in a molar ratio of 0.5:1 to 10:1, preferably from 1:1 to 3:1.

In general, the reaction takes place in a melt and is effected at an elevated temperature, preferably in the range of from 60 to 170° C., more preferably from 80 to 110° C.

The reaction time usually is chosen in the range of from 5 minutes to 40 hours, preferably from 5 minutes to 12 hours, more preferably from 3 to 5 hours.

As a rule, the reaction time and temperature are chosen so as to achieve a suitable balance between length of reaction and the amount of rhodamine type side products that are produced.

In a preferred embodiment a reaction temperature of in the range of from 80 to 110° C. and a reaction time in the range of from 3 to 5 hours are chosen.

After the reaction, either after step C, but before step D or after step D, but before step E, usually a diluent is added to the reaction mixture. According to own observations, the reaction does not continue after the addition of the diluent. Preferably, the diluent is added after step C and before step D. However, it is also possible to adjust the temperature stepwise and to add the diluent during one of the steps or to adjust the temperature continuously and to add the diluent during this adjustment process.

The weight ratio of diluent to m-amino phenol II usually is chosen in the range of 0.01:1 to 10:1, preferably in the range of from 0.25:1 to 3:1.

As diluent organic solvents and ionic liquids can be used.

As organic solvents aromatic hydrocarbons of 6 to 10 carbon atoms such as benzene, toluene or xylene, aliphatic hydrocarbons of 8-12 carbons such as octane, isooctane, or decane, cycloaliphatic hydrocarbons of 5 to 8 carbons, wherein the aromatic and cycloaliphatic hydrocarbons can be halogenated, halogenated aliphatic hydrocarbons of 2 to 8 carbons, such as perclene, chlorobenzene or dichlorobenzene, ethers such as C₄-C₆cyclic ethers like tetrahydrofuran, di-(C₂-C₆alkyl) ether like dibutyl ether or diphenylether, or C₁-C₄alkanols, among which are especially preferred C₆-C₁₀aromatic hydrocarbons such as toluene or ethers, C₁-C₄alkanols such as methanol, ethanol, propanols such as isopropanol or butanols such as n-butanol. It is also possible to use mixtures thereof and aqueous mixtures with the abovementioned organic solvents.

Ionic liquids are well known in the art and Chem. Rev. 1999, 99, 2071-2083 is hereby incorporated by reference as an example.

Before or after the addition of the diluent, as a rule the temperature of the reaction mixture is adjusted to allow for an efficient separation.

Usually, the temperature to which the reaction mixture is adjusted is chosen in the range of from 0 to 60° C., most preferably from 20 to 40° C. The adjustment can be carried out stepwise or continuously. E.g. a stepwise procedure would be preferred in a case, where a diluent is added which has a boiling point lower than the reaction temperature. In such a case the temperature preferably is adjusted to a temperature range below the boiling point of the diluent and then adjusted to the final desired temperature range as defined above.

According to the invention, the liquid phase is then separated from the solid phase of the reaction mixture usually by measures known to the skilled artisan such as e.g. filtration, centrifugation, decantation or other suitable methods of separation). The solid phase contains crude keto acid I.

In a preferred embodiment of this invention, the thus obtained solid phase can be washed with usual organic solvents in known manners, and then dried afterwards.

The liquid phase, which usually may contain keto acid I and excess m-amino phenol II, is recycled, i.e. used as starting material or as part of starting material in another cycle. For this reason phthalic anhydride and/or m-amino phenol II are added to the liquid phase in order to obtain a molar ration of phthalic anhydride and m-amino phenol in the range of from 0.5:1 to 10:1, preferably from 1:1 to 3:1.

The diluent is removed preferably by distillation either at atmospheric pressure, or under reduced pressure, prior to recycle.

In another embodiment of this invention the keto acid I may be extracted from the reaction mixture with an aqueous alkaline solution such as sodium hydroxide or potassium carbonate and then precipitated with acid. An analogous procedure is known from e.g. JP-A2 49080049.

In another embodiment, the keto acid I may be transferred into the corresponding alkali metal salt, for example lithium, sodium or potassium salt, most preferably sodium salt, followed by the isolation of this salt and then precipitation of it with aqueous acid, for example hydrochloric or sulphuric acid. An analogous procedure is known from e.g. JP-A2 62070350.

The keto acid I obtained according to the inventive methods—if desired—can be dissolved or slurried under heating in an organic solvent, for example an aliphatic alcohol of 1-8 carbons such as methanol, ethanol, n-propanol, isopropanol, butanol such as n-butanol, n-pentanol, n-hexanol, n-heptanol, n-octanol, preferably 1-4 carbons such as methanol, ethanol, n-propanol, isopropanol or butanols such as n-butanol. Then, usually, a crystallisation is carried out.

A preferred embodiment relates to the use of a mixture of C₁-C₈alcohol as decribed above with water, or a mixture of such a C₁-C₈alcohol with a hydrocarbon solvent, preferably an aromatic hydrocarbon of 6-10 carbons such as toluene or xylene, or an aliphatic hydrocarbon of 5-10 carbon atoms such as pentane, hexane or heptane. Such a procedure is described in detail e.g. in EP-A 858,993.

Should a further purification be desired, the crystals obtained according to the above described process may be dissolved or slurried with a C₁-C₈alcohol at atmospheric or elevated pressure (100 kPa to 300 kPa) at an elevated temperature (usually in the range of from 50 to 150° C. and then the solution or slurry can be cooled to cause crystallisation of the purified keto acid I to occur.

A further embodiment of the present invention relates to a method of producing a fluoran compound which comprises reacting a keto acid with a substituted phenol derivative in ways known in the art, e.g. described in U.S. Pat. No. 5,166,350, wherein the keto acid is produced according to the inventive process.

Another embodiment of the present invention relates to a heat-sensitive recording material, comprising a colour former, a sensitiser and a developer, wherein the colour former is a fluoran compound produced according to the above-described process. The manufacture of heat-sensitive materials is well known in the art and described e.g. in WO 00/26037.

As above set forth the reaction of the m-aminophenol derivative with phthalic anhydride is carried out in the absence of an organic solvent thus reducing the economic and environmental costs of the process, reducing reaction times and increasing yields.

### EXAMPLES

### Example 1

88.40 g (0.4 mol) of N,N-dibutylaminophenol and 42.32 g (0.29 mol) phthalic anhydride are placed in a reactor and stirred. The reaction mass is heated to 90 to 95° C. and stirred at this temperature for 4 hours. Liquid chromatographic analysis showed 90% conversion to the keto acid. Toluene (69.2 g) is added at 95° C. and the reaction mixture is stirred for 1 hour at this temperature before being cooled slowly to 20° C. The reaction does not proceed any further after the addition of toluene. The product, 4-N,N-dibutylamino-2-hydroxy-2′-carboxy benzophenone is isolated by filtration and washed with toluene. After drying, a crude yield of 77.97 g (73.9%) is obtained. The product contains 0.05% rhodamine as determined by HPLC.

### Example 2

The mother liquors obtained from example 1 are evaporated to give a residue containing N,N-dibutylaminophenol and 4-N,N-dibutylamino-2-hydroxy-2′-carboxy benzophenone. 63.14 g (0.29 mol) N,N-dibutylaminophenol and 42.32 g (0.29 mol) phthalic anhydride are added and the reaction mass is warmed to 90 to 95° C. and maintained at this temperature for 4 hours. Toluene (69.2 g) is added at 95° C. and the reaction mixture is stirred for 1 hour at this temperature before being cooled slowly to 20° C. The reaction does not proceed any further after the addition of toluene. The product, 4-N,N-dibutylamino-2-hydroxy-2′-carboxy benzophenone is isolated by filtration and washed with toluene. After drying, a crude yield of 92.85 g (88.0%) is obtained. The product contains 0.11% rhodamine as determined by HPLC.

### Example 3

The mother liquors obtained from example 2 are evaporated to give a residue containing N,N-dibutylaminophenol and 4-N,N-dibutylamino-2-hydroxy-2′-carboxy benzophenone. 63.14 g (0.29 mol) N,N-dibutylaminophenol and 42.32 g (0.29 mol) phthalic anhydride are added and the reaction mass is warmed to 90 to 95° C. and maintained at this temperature for 4 hours. Toluene (69.2 g) is added at 95° C. and the reaction mixture is stirred for 1 hour at this temperature before being cooled slowly to 20° C. The reaction does not proceed any further after the addition of toluene. The product, 4-N,N-dibutylamino-2-hydroxy-2′-carboxy benzophenone is isolated by filtration and washed with toluene. After drying, a crude yield of 92.85 g (88.0%) is obtained. The product contains 0.17% rhodamine as determined by HPLC.

### Example 4

100.00 g (0.27 mol) of crude 4-N,N-dibutylamino-2-hydroxy-2′-carboxy benzophenone and 150 g of methanol are charged to a glass pressure vessel. After sealing the vessel the contents are heated to 90 to 95° C. and stirred for 45 minutes. After cooling to 20° C., the product is filtered and washed with methanol. The resultant crystals are dried to provide 92.12 g (92%) of high purity 4-N,N-dibutylamino-2-hydroxy-2′-carboxy benzophenone containing no rhodamine by HPLC. A further 5.10 g (5%) of pure keto acid are obtained by evaporating the methanol liquors to 25% of their original volume and filtering the precipitated solids.

### Example 5

88.40 g (0.4 mol) of N,N-dibutylaminophenol and 42.32 g (0.29 mol) phthalic anhydride are placed in a reactor and stirred. The reaction mass is heated to 90 to 95° C. and stirred at this temperature for 4 hours. Liquid chromatographic analysis shows 90% conversion to the keto acid. 63.14 g (0.29 mol) of N,N-dibutylaminophenol is added at 95° C. and the reaction mixture is cooled slowly to 20° C. The product, 4-N,N-dibutylamino-2-hydroxy-2′-carboxy benzophenone is isolated by filtration and washed with toluene. The liquors are then evaporated, treated with 42.32 g (0.29 mol) of phthalic anhydride and the reaction mass is warmed to 90 to 95° C. and maintained at this temperature for 4 hours. The process is repeated giving the following average crude yields:

1^{st} run, yield=73.9%; 2^{nd} run, yield=81%; 3^{rd} run, yield=83.3%

### Example 6

88.40 g (0.4 mol) of N,N-dibutylaminophenol and 42.32 g (0.29 mol) phthalic anhydride are placed in a reactor and stirred. The reaction mass is heated to 133° C. and stirred at this temperature for 10 minutes. After cooling to 85 to 90° C., toluene (69.2 g) is added and the reaction mixture is stirred for 30 minutes at this temperature before being cooled slowly to 20° C. The reaction does not proceed any further after the addition of toluene. The product, 4-N,N-dibutylamino-2-hydroxy-2′-carboxy benzophenone is isolated by filtration and washed with toluene. After drying, a crude yield of 69.95 g (66.3%) is obtained. The product contains 0.15% rhodamine as determined by UV absorbance.

### Example 7

41.2 g (0.3 mol) of N,N-dimethylaminophenol and 22.2 g (0.15 mol) phthalic anhydride are placed in a reactor and stirred. The reaction mass is heated to 90 to 95° C. and stirred at this temperature for 5 hours. Methanol (24 g) is added at 80° C. and stirred under reflux for 1 hour at 63-68° C. The reaction mixture is cooled slowly over 2 hours to 20° C., then stirred for 30 minutes. The product, 4-N,N-dimethylamino-2-hydroxy-2′-carboxy benzophenone is isolated by filtration and washed with methanol. After drying, a crude yield of 42.8 g (58% theory) is obtained. The product contains 0.02% rhodamine as determined by O.D.

### Example 8

49.6 g (0.3 mol) of N,N-diethylaminophenol and 22.2 g (0.15 mol) phthalic anhydride are placed in a reactor and stirred. The reaction mass is heated to 90 to 95° C. and stirred at this temperature for 5 hours. Toluene (17.3 g) is added and the reaction mixture is cooled to 60° C. and stirred at this temperature for 1 hour. The reaction mixture is further cooled over 30 minutes to 30° C., treated with toluene (30.2 g) and stirred for 12 hours at 20° C. The product, 4-N,N-diethylamino-2-hydroxy-2′-carboxy benzophenone is isolated by filtration and washed with methanol. After drying, a crude yield of 28.0 g (59.6% theory) is obtained. The product contains 0.07% rhodamine as determined by O.D.

### Example 9

165.6 g (0.8 mol) of N-isoamyl-N-ethylaminophenol and 84.6 g (0.57 mol) phthalic anhydride are placed in a reactor and stirred. The reaction mass is heated to 90 to 95° C. and stirred at this temperature for 4 hours. Tetrachloroethane (378 g) and sodium hydroxide (aq) (50%, 128 g) is added and the mixture stirred for 30 minutes at 50-60° C. After phase separation, the aqueous layer is treated with water (237 g), tetrachloroethane (1007 g) and hydrochloric acid (189 g). After stirring at 50-60° C. for 30 minutes the aqueous layer is removed. The organic layer is then mixed with water (394 g), sodium hydroxide (213 g) and the tetrachloroethane is removed by steam distillation. The remaining aqueous solution is adjusted to pH 2-3 with sulfuric acid (20%, 200 g) to yield a pink solid which is filtered at 20° C. After drying the crude yield is 108.3 g (53.4% theory). The product, 4-(N-isoamyl-N-ethyl)amino-2-hydroxy-2′-carboxy benzophenone, contains 0.1% rhodamine as determined by OD.

### Example 10

178 g (0.8 mol) of N,N-dibutylaminophenol and 84.6 g (0.58 mol) phthalic anhydride are placed in a reactor and stirred. The reaction mass is heated to 90 to 95° C. and stirred at this temperature for 4 hours. Methanol (138 g) is added at 80° C. and stirred under reflux for 2 hours at 63-68° C. The reaction mixture is cooled slowly over 2 hours to 20° C., then stirred for 30 minutes. The product, 4-N,N-dibutylamino-2-hydroxy-2′-carboxy benzophenone is isolated by filtration and washed with methanol. After drying, a crude yield of 156.6 g (74.2% theory) is obtained. The product contains 0.1% rhodamine as determined by HPLC.

1. Method of producing keto acids having the formula I 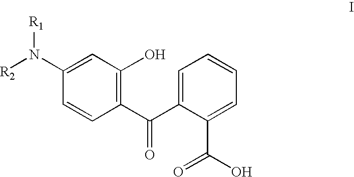
wherein R₁ and R₂ independently represent
(a) hydrogen, wherein at least one of R₁ and R₂ do not stand for hydrogen,
(b) branched or unbranched alkyl of 1-18 carbon atoms, which may be substituted by C₁-C₄alkoxy or 2- or 3-tetrahydrofuryl,
(c) a cycloalkyl of 4-8 carbon atoms,
(d) C₄-C₈cycloalkyl-C₁-C₄alkyl, or phenyl, wherein both, cycloalkyl and phenyl, may be substituted by at least one member selected from the group consisting of halogen atoms and alkyls having 1-4 carbon atoms,
(e) an aralkyl of 7-10 carbon atoms, or
(f) R₁ and R₂ together with the adjacent nitrogen atom may form a heterocyclic ring,
by reacting a m-amino phenol having the formula II 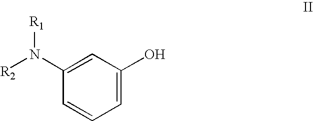
with phthalic anhydride at an elevated temperature in the absence of an organic solvent, which method comprises:
(I) mixing m-amino phenol II and phthalic anhydride in a molar ratio of from 0.5 to 10:1,
(II) melting the mixture of step (I) at an elevated temperature,
(III) choosing a reaction time in the range of from 5 minutes to 40 hours,
(IV) then separating the liquid phase from the solid phase.

2. Method of producing keto acids of formula I 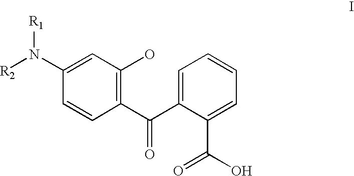
wherein R₁ and R₂ independently represent
(a) hydrogen, wherein at least one of R₁ and R₂ do not stand for hydrogen,
(b) branched or unbranched alkyl of 1-18 carbon atoms, which may be substituted by C₁-C₄alkoxy or 2- or 3-tetrahydrofuryl,
(c) a cycloalkyl of 4-8 carbon atoms,
(d) C₄-C₈cycloalkyl-C₁-C₄alkyl, or phenyl, wherein both, cycloalkyl and phenyl, may be substituted by at least one member selected from the group consisting of halogen atoms and alkyls having 1-4 carbon atoms,
(e) an aralkyl of 7-10 carbon atoms, or
(f) R₁ and R₂ together with the adjacent nitrogen atom may form a heterocyclic ring,
by reacting a m-amino phenol having the formula II 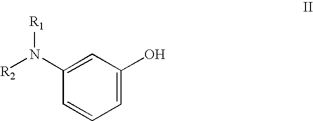
with phthalic anhydride at an elevated temperature in the absence of an organic solvent, which method comprises the following reaction cycle:
(A) mixing m-amino phenol II and phthalic anhydride in a molar ratio of from 0.5 to 10:1,
(B) melting the mixture of step (A) to an elevated temperature,
(C) choosing a reaction time in the range of from 5 minutes to 40 hours,
(D) adjusting the temperature of the reaction mixture for effective separation,
(E) then separating the liquid phase from the solid phase, optionally washing the solid phase comprising keto acid I with an organic solvent and then drying it,
(F) adding phthalic anhydride and/or m-amino phenol II to the separated liquid phase of step (E), wherein the molar ratio is from 0.5 to 10:1,
(G) using the obtained mixture of step (F) as starting material or as part of starting material of step (B) after removing the diluent,
wherein either after step C, but before step D or after step D, but before step E, a diluent is added to the reaction mixture.

3. Method of producing a fluoran compound which comprises reacting a keto acid with a substituted phenol derivative, wherein the keto acid is produced according to claim 1.

4. Heat-sensitive recording material, comprising a color former, a sensitizer and a developer, wherein the color former is a fluoran compound produced according to claim 3.
